(19) United States
(12) Patent Application Publication
(10) Pub. No.: US 2026/0151542 A1
(43) Pub. Date: Jun. 4, 2026

(54) HYALURONIC-ACID-BASED HYDROGEL WITH ARTICULAR CARTILAGE CONSTRUCT FOR SURGICAL REPAIR OF DAMAGED ARTICULAR CARTILAGE

(71) Applicant: The Stone Research Foundation for Sports Medicine and Arthritis, San Francisco, CA (US)

(72) Inventor: Kevin R. Stone, San Francisco, CA (US)

(21) Appl. No.: 19/406,856

(22) Filed: Dec. 2, 2025

Related U.S. Application Data
(60) Provisional application No. 63/727,113, filed on Dec. 2, 2024.

(51) Int. Cl. A61L 27/52; A61L 27/20; A61L 27/36; A61L 27/38; A61L 27/48; A61L 27/54

(52) U.S. Cl. CPC A61L 27/52; A61L 27/20; A61L 27/3608; A61L 27/3612; A61L 27/3654; A61L 27/3834; A61L 27/3852; A61L 27/48; A61L 27/54; A61L 2300/414; A61L 2400/06; A61L 2430/06

(57) ABSTRACT

A paste graft construct for repairing cartilage defects includes a prepared bone and cartilage plug that is ground into a paste and combined with a hyaluronic acid-based hydrogel. The combined paste and hydrogel may also be mixed with mesenchymal stem cells (MSCs) and/or birth tissue such as Wharton's jelly. The bone and cartilage plug may be prepared by smashing or cutting.

### CROSS REFERENCE TO RELATED APPLICATIONS

This application claims the benefit of priority to U.S. Provisional Application Ser. No. 63/727,113 filed Dec. 2, 2024, incorporated herein by reference.

### BACKGROUND

Traumatic injury and cartilage degeneration are seen as causes for the development of osteoarthritis (OA) and, as demographics change, the number of patients with cartilage damage can be expected to rise significantly. Every year in the U.S, there are more than 600,000 traumatic injuries to knee articular cartilage, and approximately half of these require surgery to repair. In addition, older individuals face degeneration of their knee cartilage. Articular cartilage damage ranges from mild and asymptomatic, to extensive and severely affecting function. Over time, damage frequently progresses from less to more severe pathology. Cartilage defects are common, have a range of severity, and frequently are present in relatively young individuals. Current conventional treatment options for cartilage defects include debridement, shaving and abrasion arthroplasty, subchondral drilling, microfracture, allograft transplantation, autograft implantation, and autologous cell implantation. Each treatment may have value but has also demonstrated significant limitations. There remains a need for improvement in the treatment modalities for the range of cartilage injuries and chronic articular degeneration.

Presently there are few biologic therapies available for cartilage repair. Microfracture is a standard surgical procedure where the subchondral bone of an articular defect is perforated by a surgical pick, so that bone marrow exudes and coagulates in the defect site and provides a source of cells for tissue repair. The repair tissue typically is fibrocartilage, which may be functional for several years but then frequently fails. MACI™ is an autologous cell transplantation product of cultured chondrocytes on porcine collagen membrane. It is a primary commercial tissue-engineered product currently approved in the US for repair of articular defects in the knee. MACI typically requires two surgeries separated by approximately 6 weeks, where the first surgery removes some cartilage from the patient's knee joint to provide a source of chondrocytes that are then cultured in vitro to increase the cell number. The cell preparation is seeded onto a porcine collagen membrane, and, in a second surgery, the construct is placed into the articular defect and fixed in place with fibrin glue. The repair is expected to be functional after more than 6 months and is expensive, with an estimated cost of $20-40K. Alternative approaches utilize allogeneic cartilage therapies, but those are also costly and are require significant pre-planning and complex execution. The high costs of those therapies combined with uncertain outcomes exclude a large proportion of the patient population from access.

Of those affected by osteoarthritis, the worst affected communities, both in terms of prevalence and treatment efficacy, are African Americans, females, sports athletes and those with manual occupations. African Americans are more susceptible to progressive knee osteoarthritis and have greater pain and functional limitations, even post-operatively, related to osteoarthritis. Women have a higher prevalence of osteoarthritis, especially in the over 50 age group. They may not be a candidate for a Total Knee Arthroplasty (“TKA”) due to age, and alternative treatment modalities are limited. The incidence of osteoarthritis is higher in those with occupations that require physical activity, and osteoarthritis has a greater negative impact to workers' livelihood in those occupations. Procedures such as TKA and MACI not only require significant time off work for the surgeries themselves, but also require lengthy rehabilitation and may place a limit on when workers are able to perform work involving physical activity.

Cartilage-Bone Paste Grafting (PG) has been used to repair damaged articular (joint) cartilage either as a result of injury or osteoarthritis. Clinical outcome studies up to 23 years after treatment have shown success in long term repair of the articular defect sites. However, significant issues remain with the procedure and adoption of the procedure has been limited.

Hyaluronic acid, also called hyaluronan, is an anionic, nonsulfated, glycosaminoglycan distributed widely throughout connective, epithelial, and neural tissues as part of the intracellular matrix of humans and many other mammals. Human hyaluronic acid typically is a large molecule polymer of disaccharides, which are composed of D-glucuronic acid and N-acetyl-D-glucosamine, linked via alternating β-(1→4) and β-(1→3) glycosidic bonds. Hyaluronic acid can be 25,000 disaccharide repeats in length. Polymers of hyaluronic acid can range in size from 5,000 to 20,000,000 Da in vivo. Commercially marketed hyaluronic acid may be extracted from animal tissues or synthesized by certain bacterial cultures.

Hyaluronic acid is an important component of normal articular cartilage, where it is present as a coat around each cell (chondrocyte). When aggrecan monomers bind to hyaluronan in the presence of HAPLN1 (hyaluronic acid and proteoglycan link protein 1), large, highly negatively charged aggregates form. These aggregates imbibe water and are responsible for the resilience of cartilage (its resistance to compression). The molecular weight (size) of hyaluronan in cartilage decreases with age, but the amount increases. Thiolating hyaluronic acid adds sulfur-based functional groups to hyaluronic acid thus allowing the hyaluronic acid to cross link and form firmer gels. Other functional groups that can be added to activate hyaluronic acid that can be used to permit crosslinking include sugars such as Dextran and polar side chains and groups such as hydroxyl groups, amides, and amines.

### SUMMARY OF THE INVENTION

Autologous cells and tissue support a functional articular cartilage repair construct in the form of an improved paste graft to provide improved repair of articular cartilage. The paste graft may comprise a conventional type defined as articular cartilage and underlying bone mechanically smashed into a bone-cartilage paste as augmented by birth tissue such as amniotic fluid, amniotic membranes, or Wharton's Jelly, with or without the addition of autologous or allograft mesenchymal stem cells, and with or without added hyaluronic acid-based hydrogel. For purposes of this document, the hyaluronic acid-based hydrogel comprises thiolated or other activated hyaluronic acid.

The so-disposed birth tissue-augmented paste graft repair construct, when implanted into a defect, resolves small and large cartilage defects with complex shapes. The repair construct is implantable in a patient, and preferably adapted for application with a single surgical procedure. In its natural state, the birth tissue-augmented cartilage-bone paste is sticky and generally stays in place after in situ impaction. In some embodiments, glycerol is added to the paste graft, providing enhanced “stickiness” to the impacted paste graft. Increased stickiness characterizing the birth tissue component improves the handling characteristic when placing the graft into the defect and improve cellular migration into the repair tissue. In embodiments the repair construct is delivered using arthroscopic or open surgery. Application of the repair construct in a patient is characterized by modest cost, off the shelf nature and wide availability.

A base for the articular cartilage paste graft construct of this disclosure, is best derived from the joints of respective patients having articular cartilage defects or arthritis, although such paste grafts may be from other persons, or even a non-immunogenic xenograft. The paste grafts of this disclosure result in consistent and long-term functional repair. With the disclosed surgical approach described below, issues are overcome regarding fixation of the paste graft within the joint.

### BRIEF DESCRIPTION OF THE DRAWINGS

FIG. 1 is a flowchart of a paste graft procedure using a hyaluronic acid-based hydrogel with articular cartilage construct, in embodiments.

FIG. 2 shows a birth tissue-augmented scaffold/construct, in embodiments.

FIG. 3 is a photograph of histology from an experiment with a rabbit using a paste graft comprising prepared cartilage with bone, hyaluronic acid-based hydrogel, and mesenchymal stem cells (MSCs), in embodiments

FIG. 4 illustrates histology from an experiment with a rabbit using a paste graft similar to that of FIG. 3 but without the MSCs.

FIG. 5 illustrates histology from a control experiment with a rabbit without paste grafting.

FIG. 6 illustrates comprehensive defect repair findings of a study of the safety and effectiveness of autologous tissue-hydrogel and tissue-hydrogel MSC constructs, in embodiments.

FIG. 7 is a graph illustrating quantitative assessment of the histology to compute percent fill, in embodiments.

FIG. 8 is a graph illustrating quantitative assessment of the histology to compute percent attachment, in embodiments.

### DETAILED DESCRIPTION OF THE EMBODIMENTS

Autologous cells and tissue support a functional articular cartilage repair construct in the form of a novel paste graft. For example, the paste graft may comprise articular cartilage and underlying bone mechanically smashed into a bone-cartilage paste augmented by hyaluronic acid-based hydrogel. In embodiments, the paste graft may also include autologous or allograft mesenchymal stem cells. In a further embodiment, any of the paste grafts described herein may also include birth tissue such as Wharton's jelly.

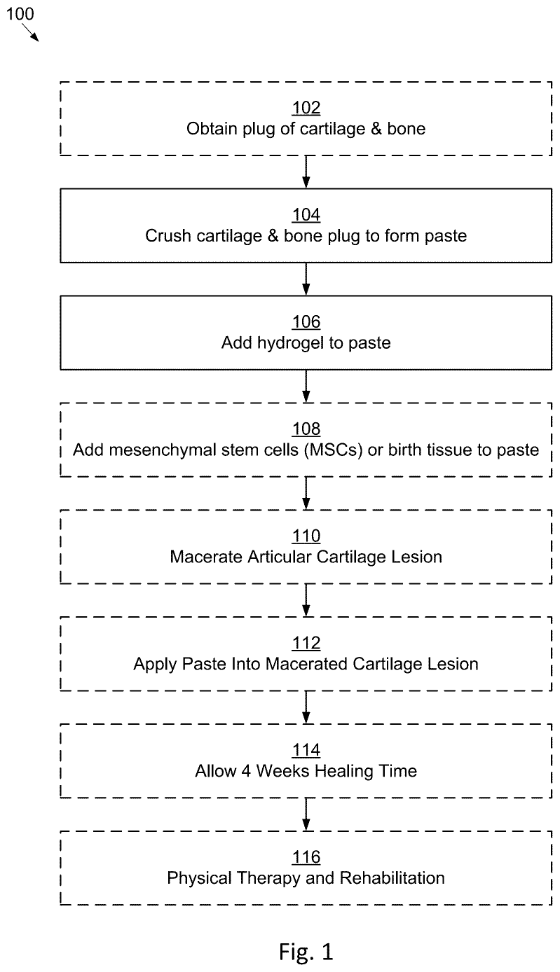

FIG. 1 is a flowchart illustrating a method 100 for surgical repair of damaged articular cartilage using an articular cartilage construct with hyaluronic-acid-based hydrogel. Method 100 includes steps 104 and 106. In embodiments, method 100 includes steps 104, 106 and 108. In further embodiments, method 100 also includes steps 102, 110, 112, 114 and 116.

Step 102 includes obtaining a plug of cartilage and bone. In an example of step 102, a plug of bone with overlying cartilage is obtained from a patient using an arthroscopic tool. A representative surgical approach morselizing a lesion to bleeding bone and harvesting of articular cartilage and cancellous bone from the intercondylar notch. In embodiments, the removed plug has a diameter of approximately 8 millimeters and is approximately 15 millimeters long. The plug has an approximate volume of one milliliter. A base for the articular cartilage paste graft construct is best derived using autograft bone and cartilage from joints of the patient requiring the graft to repair articular cartilage defects or arthritis, although in other embodiments such paste grafts may be an allograft from other persons, or even a non-immunogenic xenograft. The paste grafts of this disclosure result in consistent and long-term functional repair.

Step 104 includes preparing the plug by crushing, thinly slicing, and/or grinding to form a paste while avoiding excessive temperatures. In an example of step 104, the plug is crushed by smashing only using an impactor, by a Cartilator, a device that processes tissue into a fine, homogenously cut mixture, or by a Rongeur Cutter. The Cartilator is described in more detail in U.S. Ser. No. 19/181,434 filed Apr. 17, 2025, and titled “Device for Slicing and Crushing Bone.” incorporated herein by reference.

Step 106 includes adding a hydrogel to the paste. In an example of step 106, the hydrogel is a hyaluronic acid-based hydrogel. In a further example of step 106, approximately 1.25 ml of hyaluronic acid-based hydrogel is added for each milligram of cartilage and bone paste. In embodiments, the hydrogel is based on thiolated hyaluronic acid.

In an alternative embodiment an alternative hydrogel includes destran-tryamine (Dex-TA) plus hyaluronic acid-tyramine is used in place of the thiolated hyaluronic-acid-based hydrogel previously described.

In other embodiments, a polypeptide or protein-based hydrogel, a polysaccharide-based hydrogel, or a synthetic hydrogel having stickiness and viscosity similar to thiolated hyaluronic acid-based gel may be substituted for the thiolated hyaluronic acid-based gel herein disclosed as a component of the paste graft. It is important that the gel used be biocompatible as cell growth is expected. The gel should also be one that does not activate the immune system as immune reactions would inhibit cell growth.

Step 108 includes adding mesenchymal stem cells to the paste. In an example of step 108, the resulting paste after step 108 is 40-50% prepared bone-cartilage from a fresh bone-cartilage plug, 50-60% hyaluronic acid-based hydrogel, and mesenchymal stem cells (MSCs), where the MSCs and hyaluronic acid-based gel are blended into the fresh, prepared, bone-cartilage plug to form the paste. In embodiments, the MSCs are derived from birth products. The MSCs are in some embodiments are autograft mesenchymal stem cells derived through tissue culture from samples taken from the patient being treated, or in other embodiments are allograft stem cells derived from birth tissues. In embodiments, 1,000,000 to 10,000,000 MSCs are used.

In a further example of step 108, one milliliter of the Wharton's jelly, or a synthetic substitute having many of the same growth factors, is added to one milliliter of the prepared bone and cartilage paste mixed with hyaluronic acid-based hydrogel then mixed to form a paste. In embodiments, the resulting paste includes 1.25 microliters of a 50-50 Wharton's jelly-hyaluronic-acid-based hydrogel for each milligram of prepared bone and cartilage.

In step 110, the articular cartilage lesion is macerated. In an example of step 110, loose fragments of cartilage are removed from the cartilage lesion area of the patient, the cartilage lesion area is morselized leaving a defect depression or cavity.

In step 112, the paste from step 106 or 108 is injected into the defect area leaving the defect depression or cavity about half full of the paste; in embodiments injection into the defect depression or cavity is done arthroscopically, in alternative embodiments an open procedure is used.

In step 114, the surgical wound is then closed and the patient is prescribed four weeks of non-load-bearing healing time.

In step 116, after the healing time, the patient performs physical therapy and rehabilitation.

When using hyaluronic acid hydrogel without birth products, 1.25 μL-1.67 μL hydrogel/mg tissue provided for the best handling characteristics with a putty-like consistency.

The so-disposed birth tissue-augmented paste graft repair construct, inter alia, resolves small and large cartilage defects with complex shapes. The repair construct is implantable in a patient, and preferably adapted for application with a single surgical procedure. In its natural state, the birth tissue-augmented cartilage-bone paste is sticky and generally stays in place after in situ impaction. In some embodiments, glycerol is added to the paste graft in addition to the birth tissue, providing enhanced “stickiness” to the impacted paste graft. Increased stickiness characterizing the birth tissue component improves the handling characteristic when placing the graft into the defect and improve cellular migration into the repair tissue. In embodiments the repair construct is delivered into the defect using arthroscopic or open surgery. For example, a solution of 10% glycerol may be blended into the paste.

In accord with the articular cartilage paste graft construct disclosed herein, an articular cartilage defect to be repaired is identified, and the area defining the defect is debrided and morselization is performed. Functional articular cartilage from a non-load bearing region of a joint, preferably a joint of the patient, is harvested together with subchondral bone, and prepared by crushing, grinding, or slicing into a paste using a mechanical device. A hyaluronic acid-based hydrogel is mixed with the harvested and prepared functional articular cartilage-bone paste. The resultant paste composite is applied to the defect site together with morselized lesion to which the paste naturally adheres. The birth tissue-augmented graft supports retention of the paste within the defect site. Local bone marrow mesenchymal stromal cells, together with the paste graft, extracellular matrix, chondrocytes and bone cells, combine to stimulate and support formation of the new articular cartilage. MSCs and/or birth tissue (for example, and in some embodiments, Wharton's jelly) may also be mixed with the harvested and prepared functional articular cartilage-bone-hydrogel paste prior to it's application to the defect site.

Once implanted, the birth tissue-augmented paste graft composite (or construct) in situ generates cells that proliferate and secrete a cartilage extracellular matrix. That matrix forms a functional articular cartilage construct that is integrated with the at least in part surrounding articular cartilage of the joint. Efficacy of that functional articular cartilage construct is defined by both (1) the regeneration of articular repair tissue over the exposed bone and (2) relief of pain.

A method of delivery to a patient of the birth tissue-augmented paste graft composite, is by arthroscopic surgery effecting impactions of the composite to the defect site, followed by relying on the stickiness of the birth tissue-augmented or hyaluronic-acid-based gel augmented paste graft composite to hold the paste graft composite in place. The biocompatibility risk associated with the birth tissue-augmented paste graft composite, is in the combination of the paste graft with the birth tissues. For optimum functionality, relative “dose” is in terms of the concentration of paste graft with-in the birth tissues is 0.25 cc to 8×15 mm cylinder (core measured prior to compaction into the paste graft).

In preparation of the constructs of the present disclosure, paste graft preparation performed pursuant to the methodology described above, resulted in increased mobility of chondrocytes by matrix disruption without significant loss of cell viability. The impaction procedure stimulated chondrocyte proliferation resulting in a cellular response to reestablish native extracellular matrix. Analysis of gene expression supports a regenerative process of cartilage tissue formation and contradicts long-held beliefs that impaction trauma leads to immediate cell death. This mechanism of action translates into clinical benefit for patients with cartilage damage.

The disclosure herein sets forth a surgical Mesenchymal Stem Cell (MSC)-based autograft and allograft therapy to repair focal and large articular cartilage defects that would preferably be a single procedure, easy and reproducible to use, ‘off the shelf’ and characterized by modest cost.

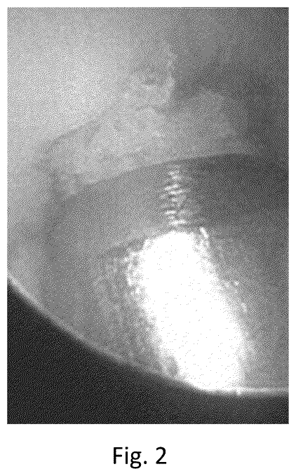

FIG. 2 shows an exemplary birth tissue-augmented articular cartilage autologous cellular repair construct in accord with the disclosure. In view of the above-noted prior art studies, but pursuant to the methods of this disclosure relating to utilization of birth tissue, an articular cartilage repair device comprises a birth tissue-augmented articular cartilage autologous cellular repair (paste graft) construct formed in vivo or in vitro, and for amplification of the repair process and for adherence to the surrounding host articular cartilage and bone. In some embodiments, glycerol is added to the paste graft, providing enhanced bonding. In some embodiments, hyaluronic-acid-based gel is added to the paste graft.

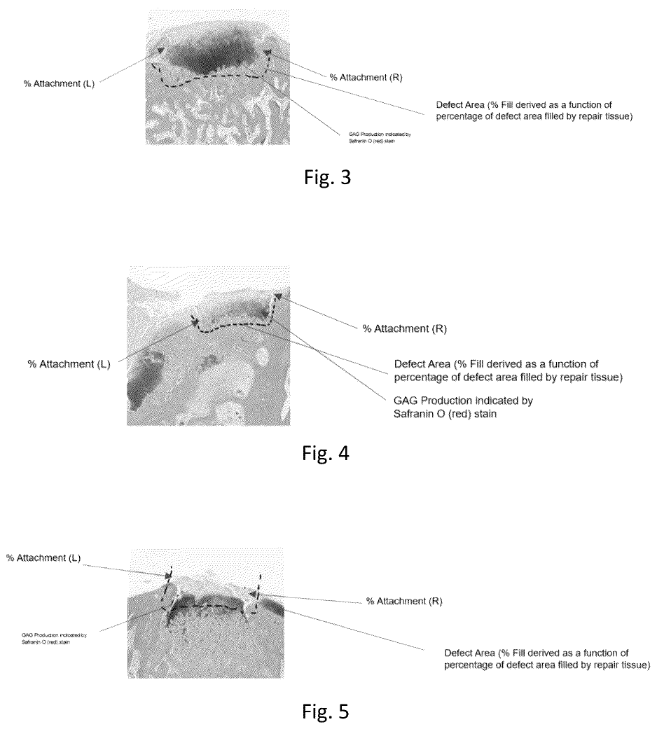

FIG. 3 is a photograph of histology from an experiment with a rabbit using a paste graft comprising prepared cartilage with bone, hyaluronic acid-based hydrogel, and mesenchymal stem cells (MSCs), in embodiments

FIG. 4 is a photograph of histology from an experiment with a rabbit using a paste graft similar to that of FIG. 3 but without the MSCs.

FIG. 5 is a photograph of histology from a control experiment with a rabbit without paste grafting.

### Paste Graft and Hydrogel-Related Studies

### In Vivo Findings: Small Animal Model (Rabbit)

The objective of the small animal model (rabbit) study was to determine the safety and effectiveness of autologous tissue-hydrogel and tissue-hydrogel-MSC constructs in a cartilage repair model. Surgery was performed on 26 rabbits (approx. 6 kg each). One articular cartilage defect (3 mm diameter, 5 mm depth) was made on the medial femoral condyle of the right knee. Defects were made with a hand drill and finished with a 3-mm diameter awl. The defects were left treated or untreated according to Table 1 (see below). Rabbits were allowed normal cage activity and euthanized at day 30. Analyses included gross imaging, histology (Safranin O/Fast Green staining), and confirmatory micro-CT.

TABLE 1

| Group | Treatment |
|---|---|
| Group A | Paste and Hydrogel |
| Group B | Paste, Hydrogel, and MSCs |
| Group C | Untreated/Empty Defect |

The study was completed with 25 of the 26 animals (Rabbit V1895 was euthanized due to a spine and nerve issue that resulted in limping and subsequent IACUC (Institutional Animal Care and Use Committee) euthanasia of the animal). One animal in Group A, and 3 animals in Group B. were excluded from analysis since the defects were positioned too close to the notch and therefore were not fully surrounded by articular cartilage. Representative results from the gross imaging, histology, and confirmatory micro-CT are displayed below per group . . .

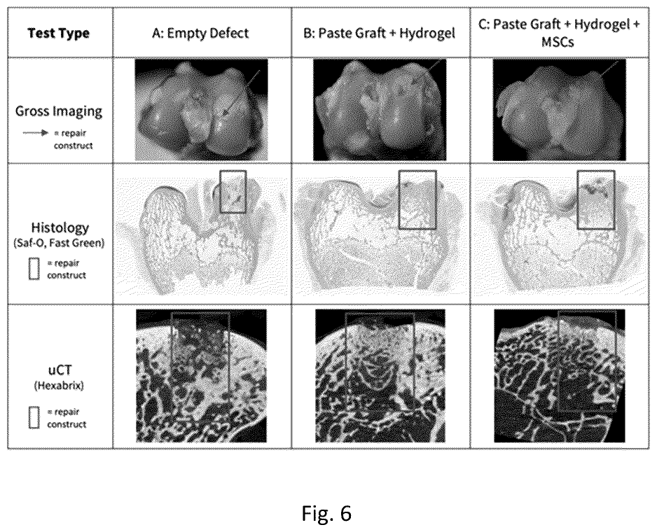

FIG. 6 illustrates comprehensive defect repair findings of a study of the safety and effectiveness of autologous tissue-hydrogel and tissue-hydrogel MSC constructs, in embodiments. The rows in FIG. 6 illustrate gross imaging, histology and uCT (computed tomography (CT) with the iodinated contrast agent ioxaglate (Hexabrix™) to non-invasively visualize and quantify tissues, especially articular cartilage. The columns in FIG. 6 illustrate Groups A, B and C from Table 1.

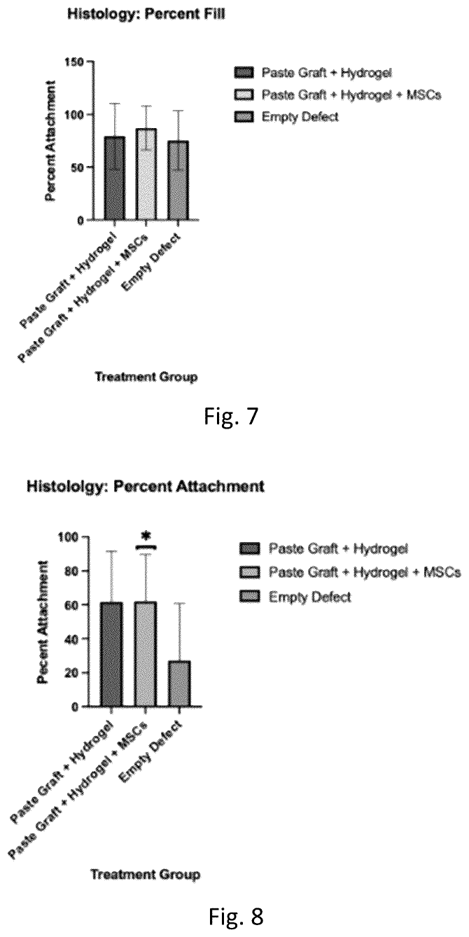

Quantitative assessment of the histology was performed, and the averages of 4 independent reviewers were used to compute percent fill as shown in FIG. 7 and percent attachment as shown in FIG. 8. Statistical analysis was performed to compare Groups B and C (Group A subgroups were too small for analysis) showed no difference in percent fill (p >0.1) and a statistically significant difference in repair tissue attachment to the surrounding tissue (p=0.03). Descriptive statistics used to characterize Group A revealed that the percent attachment was higher than that of Group C (61.7% and 27.2%, respectively).

### In Vivo Testing: Large Animal Model (Horse)

The objective of a large animal model (horse) is to assess the safety and efficacy of two methods of preparation of the articular cartilage paste graft in conjunction with the addition of hydrogel and MSCs. Ten skeletally mature horses (2-5 years of age), free of musculoskeletal disease are housed in standard stalls. For the study, horses were placed under general anesthesia, and bilateral (left and right) femoropatellar arthrotomies were carried out in each horse. A critically sized cartilage defect (15-mm in diameter) was created over the medial trochlear ridge using a round custom designed instrument and hand curette. Calcified cartilage was carefully removed. The articular cartilage paste graft technique was performed by morselizing the base of the defect with a microfracture awl. Articular cartilage and underlying bone was harvested using a custom-designed trephine and was subsequently taken outside of the knee and prepared into a paste formulated depending on the assigned treatment group. The animals were divided into 5 groups:
Treatment Groups (2 horses per group, 2 limbs/horse, 2 defects/limb):
Group A: Control-Empty Defect (N=8 defects)
Group B: Articular Cartilage Paste Graft #1 Only (N=8 defects)
Group C: Articular Cartilage Paste Graft #1+Hydrogel (N=8 defects)
Group D: Articular Cartilage Paste Graft #2+Hydrogel (N=8 defects)
Group E: Articular Cartilage Paste Graft #2+Hydrogel+MSCs (N=8 defects)

Note: Articular Cartilage Paste Graft #1 denotes smashed paste (the traditionally prepared method) and Articular Cartilage Paste Graft #2 denotes cut paste, prepared with the Cartilator device.

For Group C, the hydrogel preparation was added into the harvested and smashed paste, then impacted into the lesion. Both Groups D and E involved harvested paste processing with the Cartilator instrument, a device that processes tissue into a fine, homogenously cut mixture. Any group involving the addition of hydrogel and/or MSCs involved the preparation of the paste and the subsequent addition of hydrogel and/or MSCs at a specified ratio per unit of tissue volume. For Group A, standard microfracture technique creating 10 holes (3 mm deep) was applied as described. The joint capsule, subcutaneous tissue and skin incision were routinely sutured in 4 distinct planes. Each horse received bilateral treatments (left and right), with two defects per limb. Each treatment group subsequently included 2 defects.

Results: Midpoint arthroscopy qualitative assessments were taken to assess the following categories of defect repair: Cartilage Attachment, Bone Attachment, Firmness, Blood, Shape, Level, Color, Surface, Better/Worse, Area of Fill, and Volume of Fill.

Kruskall-Wallis testing was conducted for each metric assessed, however, no statistical significance was observed. Findings for each assessment are as follows.

Cartilage attachment: No statistical significance observed; Smashed+Hydrogel treatment group had the strongest attachment.

Bone attachment: Smashed+Hydrogel showed marginally stronger attachment; no statistical significance observed.

Firmness: Smashed+Hydrogel had slightly more similar results when compared to the surrounding healthy tissue.

Blood: No blood was observed in the defects with an exception for one defect in the Smashed Only group.

Shape: No tissue damage observed in any defect across all defects and treatment groups.

Level: No significance observed; Empty Defect was slightly recessed (mean 2.0) when compared to Smashed Only (mean 2.250) and Smashed+Hydrogel and Cartilator+Hydrogel (means 2.125). The Cartilator+Hydrogel+MSCs group was slightly more elevated than other groups (mean 3.250).

Color: No significant differences in color observed; all groups between white-yellow and white.

Surface: While no significant differences were observed, the Smashed Only group had the highest mean (2.125), and exhibited slightly more undulation than both the Smashed+Hydrogel (mean 1.75) and Cartilator+Hydrogel (mean 1.375).

Grade: Overall repair grades were assigned to groups with no statistical significance. Both Smashed+Hydrogel and Empty Defect performed the strongest with means of 2.125. Further testing and histological validation is necessary to assess strongest repair construct.

Area: All groups filled 100% of the defect area.

Volume: While no statistical significance was observed, Cartilator+Hydrogel+MSCs performed the strongest with a mean of 68.75%. Smashed+Hydrogel also performed slightly stronger (mean 62.5%) than Smashed Only (57.0%).

These summary findings suggest that while further study and histological validation are required, adding hydrogel to the smashed paste contributed to overall stronger qualitative metrics of defect repair.

### In Vitro Testing: Paste Preparation Method

The following methods of testing for paste preparation were conducted: Smashed Only (Impactor), Cartilator, and Rongeur Cut. Confocal imaging and qPCR for Aggrecan, Sox9, RunX2, Col1, and COMP were assessed at three separate time points (Day 1, Day 4, and Day 14). Principal Component Analysis (PCA) of PCR data was conducted displaying no statistically significant findings when each treatment group and time were selected for.

### In Vitro Testing: Paste Graft+Hydrogel

Three different constructs of human cartilage and bone with hydrogel were made and maintained in culture for 28 days, using cartilage and bone discarded from consented surgical patients under IRB supervision. Cell viability and metabolic activity were assessed through Alamar Blue quantitative assay. The three construct groups are as follows:
Group 1-Hydrogel+Cut Tissue,
Group 2-Hydrogel+Cut Tissue+MSCs,
Group 3-Hydrogel+Cut tissue+human MSCs+chondrogenic media.

Constructs were cultured for 28 days, and analysis of hydrogel+tissue constructs at day 0 and cultured to day 28 showed cell viability and metabolic activity increased to 192%+/−110% of the starting level (Group 1, n=12. P=0.004).

To assess differences among groups, cell viability and metabolic activity data was analyzed by one-way ANOVA with repeated measures t-test with two dependent means. Results showed that there is a statistically significant difference between treatments (p=0.01). Post-Hoc Tukeu testing showed a statistically significant difference between Groups 1 and 3 (p=0.03).

Subsequent long-term (28 day) cultures of the tissue-hydrogel constructs were assessed for matrix content, specifically S-GAG as a measure of proteoglycan content, and OH-Proline as a measure of collagen. Cartilage constructs (Group 1-Hydrogel+Tissue, Group 2-Hydrogel+Tissue+MSCs+Chondrogenic Media) were digested and analyzed for S-GAG using 1,9 dimethylmethylene blue and hydroxyproline using microtiter plate quantitative assays. S-GAG content in the group containing MSCs with chondrogenic media was elevated by 23% (p=0.02) compared to the group without MSCs and chondrogenic media. Hydroxyproline constructs showed no significant difference.

Wharton's jelly contains a broad selection of growth factors and cytokines that may affect growth and development of stem cells, see Table 2. These are listed with abbreviated names commonly used in the art of growth factors and cytokines.

TABLE 2

| | | | | | Cytokines | Cytokines |
|---|---|---|---|---|---|---|
| | | | | | associated | associated |
| | | | | | with | with |
| Growth | Immunomodulatory | Pro-inflammatory | Anti-inflammatory | Homeostatic | wound | regenerative |
| Factors | cytokines | cytokines | cytokines | cytokines | healing | properties |
| IGFBP 1 | RANTES | MCSFR | TNF-RI | TIMP-1 | ICAM-1 | GH |
| IGFBP 2 | IL-6R | MIP-1a | TNF-RII | TIMP-2 | G-CSF | |
| IGFBP 3 | IL-16 | | IL-1RA | | GDF-15 | |
| IGFBP 4 | | | | | | |
| IGFBP 6 | | | | | | |
| TGF-α | | | | | | |
| PDGF-AA | | | | | | |

As shown in Table 2, IGFPB 1 is insulin-like growth factor-binding protein 1, IGFPB 2 is insulin-like growth factor-binding protein 2, IGFPB 3 is insulin-like growth factor-binding protein 3, IGFPB 4, is insulin-like growth factor-binding protein 4, IGFPB 6 is insulin-like growth factor-binding protein 6, TGF-α is transforming growth factor alpha, PGDF-AA is platelet-derived growth factor sub chain AA. The cytokines listed for the various growth factors would be understood by one of ordinary skill in the art, for example, ICAM-1 is intercellular adhesion molecule 1, G-CSF is granulocyte colony stimulating factor, GH is growth hormone, and GDF-15 is growth/differentiation factor 1.

In an alternative embodiment, a synthetic analog of Wharton's jelly is used in place of Wharton's jelly. In particular embodiments, the synthetic analog of Wharton's jelly contains at least 5 cytokines or growth factors selected from the list of IGFPB 1, IGFPB 2, IGFPB 3, IGFPB 4, IGFPB6, ICAM-1, G-CSF, GDF-15, and GH together with a viscosity enhancing agent and an adhesion-enhancing agent.

Table 3 lists several representative recipes for the prepared bone/gel paste used for the repair construct. While experimenting, it was found that a blend with 1.25 microliters (μL) of pH-neutralized hyaluronic acid-based gel (hydrogel) per milligram (mg) of prepared cartilage/bone mix gave best physical properties, with 1.67 μL/mg prepared cartilage/bone mix gave reasonable properties. Table 3 showing proportions of each ingredient in proposed paste grafts. Actual grafts are sized appropriate to the size of the cartilage lesion being treated.

TABLE 3

| | | Hyaluronic | | | |
|---|---|---|---|---|---|
| | | acid-based | | Prepared | Wharton's |
| Recipe | MSC's | gel | | bone/cartilage | Jelly |
| 1 | no | 1.25 | uL | 1 mg | |
| 2 | no | 1.67 | uL | 1 mg | |
| 3 | no | 1 | mg | 1 mg | |
| 4 | no | 0.67 | uL | 1 mg | 0.67 uL |
| 5 | Yes | 1.25 | uL | 1 mg | |
| 6 | yes | 1.67 | uL | 1 mg | |
| 7 | no | 40-50% | | 50-60% | |
| 8 | yes | 40-50% | | 50-60% | |
| 9 | yes | 1.25 | uL | 1 mg | |

Recipe 1 gave the best results for handling of recipes 1, 2, and 3, with recipe 3 being somewhat difficult to handle.

Recipe 4 is expected to give reasonable handling characteristics while giving better cell growth than recipe 1.

Recipe 9 was found to give better cell growth than recipe 1, 2, or 3 in an experiment with rabbits.

Changes may be made in the above methods and systems without departing from the scope hereof. It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

What is claimed is:

1. A paste graft construct for repairing cartilage defects comprising prepared bone and cartilage and comprising at least one of: birth tissue, a hyaluronic acid-based hydrogel, and mesenchymal stem cells (MSCs).

2. The paste graft construct of claim 1 comprising hyaluronic-acid-based hydrogel.

3. The paste graft construct of claim 2 comprising 1.25 microliter hyaluronic-acid-based hydrogel for each milligram of prepared bone and cartilage.

4. The paste graft construct of claim 3 further comprising MSCs.

5. The paste graft construct of claim 2 further comprising birth tissue comprising Wharton's jelly.

6. The paste graft construct of claim 5 comprising 1.25 microliters of a 50-50 Wharton's jelly-hyaluronic-acid-based hydrogel for each milligram of prepared bone and cartilage.

7. A paste comprising:
40-50% prepared bone-cartilage from a fresh bone-cartilage plug;
mesenchymal stem cells (MSCs); and
50-60% hyaluronic acid-based gel;
the MSCs, hyaluronic acid-based gel, being blended into the fresh, prepared, bone-cartilage plug to form the paste.

8. The paste of claim 7 where the MSCs are derived from birth products.

9. A method of paste grafting articular cartilage defects in a patient comprising macerating a surface of an articular cartilage defect in the patient and injecting the paste of claim 7 into the articular cartilage defect.

10. The method of claim 9 where the bone-cartilage plug is autologous to the patient.

11. The method of claim 10 performed arthroscopically.

12. The method of claim 11 where the bone-cartilage plug is 8 millimeters by 15 millimeters.

13. The method of claim 12 further comprising blending up to 10% glycerol into the paste.

14. The method of claim 9 where the bone-cartilage plug is an allograft.

15. The method of claim 9 where the bone-cartilage plug is a xenograft.
